(19) United States
(12) Patent Application Publication
(10) Pub. No.: US 2021/0380967 A1
(43) Pub. Date: Dec. 9, 2021

(54) Methods of Identifying Adenosine-to-Inosine Edited RNA

(71) Applicant: Emory University, Atlanta, GA (US)

(72) Inventors: Steven D. Knutson, Atlanta, GA (US); Jennifer M. Heemstra, Atlanta, GA (US)

(21) Appl. No.: 17/425,698

(22) PCT Filed: Jan. 23, 2020
(86) PCT No.: PCT/US2020/014808

§ 371 Date: Jul. 23, 2021

Related U.S. Application Data
(60) Provisional application No. 62/795,796, filed on Jan. 23, 2019.

(51) Int. Cl. C12N 15/10; C12Q 1/6806; C12N 9/22

(52) U.S. Cl. CPC C12N 15/1013; C12Q 1/6806; C07K 2319/00; C12Y 301/21007; C12N 9/22

(57) ABSTRACT

This disclosure relates to improved methods of identifying A-to-I RNA edits in a sample. In certain embodiments, this disclosure relates to methods of purifying RNA containing an inosine base comprising the steps of: exposing an RNA sample to endonuclease V or fusion thereof and calcium ions in the absence of magnesium ions providing an RNA and endonuclease V binding complex. In certain embodiments, the methods further comprise purifying the RNA and endonuclease V binding complex from unbound RNA in the sample; separating the RNA from endonuclease V providing separated RNA; sequencing the separated RNA; and identifying positions in the RNA sequences wherein A-to-I edits occur. In certain embodiments, the RNA is derived from a cell.

### CROSS-REFERENCE TO RELATED APPLICATIONS

This application claims the benefit of U.S. Provisional Application No. 62/795,796 filed Jan. 23, 2019. The entirety of this application is hereby incorporated by reference for all purposes.

### STATEMENT REGARDING FEDERALLY SPONSORED RESEARCH OR DEVELOPMENT

This invention was made with government support under GM116991 awarded by the National Institutes of Health. The government has certain rights in the invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy and is hereby incorporated by reference into the specification. The name of the text file containing the Sequence Listing is 19090PCT ST25.txt. The text file is 3 KB, was created on Jan. 23, 2020, and is being submitted electronically via EFSWeb.

### BACKGROUND

Adenosine-to-inosine (A-to-I) RNA editing is a post-transcriptional modification catalyzed by adenosine deaminases acting on RNAs (ADARs). The reaction alters both the chemical structure and hydrogen bonding patterns of the nucleobase. A-to-I RNA editing is implicated in a variety of biological processes. Inosines preferentially base pair with cytidines, effectively recoding these sites as guanosines during PCR sequencing. Because inosine is decoded as guanosine by polymerases, raw cDNA readouts can be matched to a reference genome where A to G transitions are putative inosine sites.

A-to-I editing rates at individual sites can be highly variable or conditionally active, differing significantly across cell and tissue types, developmental states, and disease progression stages. Additionally, edited RNAs may only present in low abundance, yielding very few actual RNA-seq reads. In these cases, actual editing rates cannot be quantified, as acquiring a statistically significant number of reads would require impractically large amounts of RNA or excessively high numbers of RNA-seq reads. Limitations in accurately characterizing A-to-I sites and RNA editing activity restricts the understanding of epi-transcriptomic dynamics and regulation. Thus, there is a need for improved methods of purifying RNAs with A-to-I edits.
Nishikura reports A-to-I editing of coding and non-coding RNAs by ADARs. Nat Rev Mol Cell Biol. 2016, 17(2): 83-96.
Morita et al. report human endonuclease Vis a ribonuclease specific for inosine-containing RNA. Nature communications, 2013, 4, 2273

References cited herein are not an admission of prior art.

### SUMMARY

This disclosure relates to improved methods of identifying A-to-I RNA edits in a sample. In certain embodiments, this disclosure relates to methods of purifying RNA containing an inosine base comprising the steps of: exposing an RNA sample to endonuclease V or fusion thereof and calcium ions in the absence of magnesium ions providing an RNA and endonuclease V binding complex. In certain embodiments, the methods further comprise purifying the RNA and endonuclease V binding complex from unbound RNA in the sample; separating the RNA from endonuclease V providing separated RNA; sequencing the separated RNA; and identifying positions in the RNA sequences wherein A-to-I edits occur. In certain embodiments, the RNA is derived from a cell.

In certain embodiments, this disclosure relates to methods of isolating RNA enriched with an inosine base comprising, mixing an endonuclease V, calcium ions in the absence of magnesium ions, and an sample comprising RNA with an inosine base, under conditions such that the endonuclease V binds to the RNA forming an endonuclease V and RNA complex; purifying the endonuclease V and RNA complex; and releasing the RNA from the complex providing isolated RNA enriched with an inosine base. In certain embodiments, the endonuclease V is Escherichia coli endonuclease V. In certain embodiments, said purifying the endonuclease V and RNA complex comprises separating the endonuclease V and RNA complex from RNA that does not substantially contain an inosine base in the sample.

In certain embodiments, this disclosure relates to methods of purifying and identifying cellular RNA comprising an inosine base comprising, isolating RNA from a cell; breaking the isolated RNA into RNA fragments; mixing the RNA fragments with glyoxal providing a sample of single stranded RNA comprising an inosine base; mixing an endonuclease V, calcium ions in the absence of magnesium ions, and the sample of single stranded RNA comprising an inosine base, under conditions such that the endonuclease V bind to the RNA forming an endonuclease V and RNA complex; purifying the endonuclease V and RNA complex; and releasing the RNA from the endonuclease V, and RNA complex providing isolated cellular RNA comprising an inosine base.

In certain embodiments, this disclosure relates to a fusion peptide comprising Escherichia coli endonuclease V sequence and a heterologous peptide sequence. In certain embodiments, this disclosure relates to a cell or other expression system comprising a nucleic acid or vector disclosed herein.

In certain embodiments, this disclosure relates to kits comprising a fusion peptide comprising an endonuclease V sequence and a heterologous peptide sequence, a specific binding agent conjugated, wherein the specific binding agent binds to the heterologous peptide sequence, and a container or solution comprising calcium ion in the absence of magnesium ion.

### BRIEF DESCRIPTION OF THE SEVERAL VIEWS OF THE DRAWINGS

FIGS. 1A-H show data indicating that eEndoV recognizes inosine in ssRNA. Supplementation with Ca²⁺ enables high affinity binding and selective immunoprecipitation of inosine-containing ssRNAs.

FIG. 1A shows the chemical alterations of adenosine-to-inosine RNA editing catalyzed by ADAR enzymes.

FIG. 1B shows a crystal structure (PDB 2W35) of eEndoV complexed with ssDNA, illustrating recognition of inosine in a nucleic acid substrate and Mg²⁺ positioned adjacent to cleavage site.

FIG. 1C shows an oligoribonucleotide test sequence: AAGCAGCAGGCUXUGUU AGAACAAU (SEQ ID NO: 1) with putative cleavage site (arrow) and PAGE analysis of digestion reactions with eEndoV illustrating specificity toward RNA I and confirming Mg²⁺ requirement for cleavage. Mg²⁺ or Ca²⁺ supplementation modulates eEndoV activity towards inosine-containing RNA substrates between cleavage and binding.

FIG. 1D shows an EndoVIPER schematic targeting a Cy5-labeled ssRNA using recombinant eEndoV-MBP fusion protein and anti-MBP magnetic beads.

FIG. 1E shows a representative PAGE analysis of initial (I), flow-through (FT) and eluate (E) EndoVIPER fractions, illustrating the effects of Ca²⁺ supplementation on pulldown efficiency.

FIG. 1F shows densitometric analysis of pulldown efficiency for A- and I-containing RNA.

FIG. 1G shows fold selectivity.

FIG. 1H shows data on quantification of eEndoV binding affinity towards ssRNA I and ssRNA A using MST.

FIGS. 2A-F show data indicating eEndoV binding favors ssRNA over dsRNA substrates.

FIG. 2A shows a schematic of dsRNA target annealing

FIG. 2B shows data on duplex verification by 10% native PAGE.

FIG. 2C shows data on MST analysis of eEndoV binding affinity towards dsRNA A

FIG. 2D shows data on dsRNA I targets using MST.

FIG. 2E shows representative PAGE analysis of initial (I), flow-through (FT) and eluate (E) EndoVIPER fractions when tested with various dsRNA targets.

FIG. 2F shows data from densitometric analysis of EndoVIPER efficiency for dsRNA targets.

FIGS. 3A-H show data indicating glyoxal treatment disrupts RNA secondary structure and enables unbiased pulldown of inosine in both ssRNA and dsRNA.

FIG. 3A shows a schematic of glyoxal addition to the Watson-Crick-Franklin face on guanosine residues, forming a N¹,N²-dihydroxyguanosine adduct.

FIG. 3B illustrates general reaction conditions for installation and removal of glyoxal adducts on test RNA strands.

FIG. 3C illustrates disruption of dsRNA target annealing by glyoxal treatment

FIG. 3D shows data verification by 10% native PAGE.

FIG. 3E shows data on MST analysis of eEndoV binding affinity towards glyoxal-treated dsRNA A.

FIG. 3F shows data indicating dsRNA I targets using MST.

FIG. 3G shows representative PAGE analysis of initial (I), flow-through (FT) and eluate (E) EndoVIPER fractions when tested with various glyoxal-treated dsRNA targets.

FIG. 3H shows densitometric analysis of EndoVIPER efficiency for glyoxal-treated dsRNA targets.

FIGS. 4A-G show data indicating EndoVIPER-seq enables enrichment and high-throughput analysis of A-to-I RNA editing sites.

FIG. 4A shows a schematic of EndoVIPER-seq workflow. Cellular RNA is first randomly hydrolyzed into ˜200-500 nt fragments, followed by glyoxal denaturation. A-to-I edited RNA is then enriched by eEndoV pulldown, followed by glyoxal removal, library preparation and high-throughput sequencing.

FIG. 4B shows data on the mean number of sites between duplicate input and EndoVIPER samples shows significantly increased detection of called A-to-I positions.

FIG. 4C shows merged datasets cross referenced against known databases show that detection of both novel and existing A-to-I sites is enhanced by EndoVIPER.

FIG. 4D shows box and whisker plots show that read coverages at all A-to-I editing sites (n=73,578) are significantly increased by EndoVIPER.

FIG. 4E shows editing rates.

FIG. 4F shows box and whisker plot of calculated fold enrichment at all sites

FIG. 4G shows sequence motif analysis compiled from the top 100 most enriched transcripts. Arrow denotes A/I site.

### DETAILED DISCUSSION

Before the present disclosure is described in greater detail, it is to be understood that this disclosure is not limited to embodiments described, and as such may, of course, vary. It is also to be understood that the terminology used herein is for describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods, compared to those of the corresponding compositions or methods disclosed herein.

As used herein, the term “conjugated” refers to linking molecular entities through covalent bonds, or by other specific binding interactions, such as due to hydrogen bonding and/or other van der Walls forces. The force to break a covalent bond is high, e.g., about 1500 pN for a carbon to carbon bond. The force to break a combination of strong protein interactions is typically a magnitude less, e.g., biotin to streptavidin is about 150 pN. Thus, a skilled artisan would understand that conjugation must be strong enough to bind molecular entities in order to implement the intended results.

The term “sequencing” refers to any number of methods that may be used to identify the order of nucleotides a particular nucleic acid. Methods and instrumentation for nucleic acid sequencing are known, and, in certain embodiments, the sequencing methods are not limited to the specific method, devices, or data/quality filtering utilized. Bokulich et al. report quality-filtering improves sequencing produced by Illumina GAIIx, HiSeq and MiSeq instruments. See Nature Methods, 2013, 10:57-59. Within certain embodiments, methods disclosed herein may use PCR and/or paired-end, mate-pair methods as described in Bentley et al., Nature, 2008, 456, 53-59 and Meyer et al., Nature protocols, 2008, 3, 267-278, hereby incorporated by reference.

The term “polymerase chain reaction” (“PCR”) refers to the method of K. B. Mullis U.S. Pat. Nos. 4,683,195, 4,683,202, and 4,965,188, that describe a method for increasing the concentration of a segment of a target sequence in a mixture. This process for amplifying the target sequence consists of introducing a large excess of two polynucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured, and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any polynucleotide or polynucleotide sequence can be amplified with the appropriate set of primer molecules. In particular, the amplified segments created by the PCR process itself are, themselves, efficient templates for subsequent PCR amplifications.

The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

Certain methods may utilize fluorescently labeled nucleotides attached to a growing double stranded sequence wherein the polymerization is controlled with chemical functional groups. Areas of a solid surface are enhanced with the same oligonucleotide and the fluorescently labeled nucleotide indicates which base is being added. The approach described may also be extended to other protocols, including full sequencing of intermediate sized fragments (>300 bp).

The term “specific binding agent” refers to a molecule, such as a proteinaceous molecule, that binds a target molecule with a greater affinity than other random molecules or proteins. Examples of specific binding agents include an antibody that bind an epitope of an antigen or a receptor which binds a ligand. In certain embodiments, “Specifically binds” refers to the ability of a specific binding agent (such as an ligand, receptor, enzyme, antibody or binding region/fragment thereof) to recognize and bind a target molecule or polypeptide, such that its affinity (as determined by, e.g., affinity ELISA or other assays) is at least 10 times as great, but optionally 50 times as great, 100, 250 or 500 times as great, or even at least 1000 times as great as the affinity of the same for any other or other random molecule or polypeptide.

As used herein, the term “ligand” refers to any organic molecule, i.e., substantially comprised of carbon, hydrogen, and oxygen, that specifically binds to a “receptor.” As a convention, a ligand is usually used to refer to the smaller of the binding partners from a size standpoint, and a receptor is usually used to refer to a molecule that spatially surrounds the ligand or portion thereof. However as used herein, the terms can be used interchangeably as they generally refer to molecules that are specific binding partners. For example, a glycan may be expressed on a cell surface glycoprotein and a lectin protein may bind the glycan. As the glycan is typically smaller and surrounded by the lectin protein during binding, the glycan may be considered a ligand even though it is a receptor of the lectin binding signal on the cell surface. An antibody may be considered a receptor, and the epitope may be considered the ligand. In certain embodiments, a ligand is contemplated to be a compound that has a molecular weight of less than 500 or 1,000. In certain embodiments, a receptor is contemplated to be a protein-based compound that has a molecular weight of greater than 1,000, 2,000 or 5,000. In any of the embodiments disclosed herein the position of a ligand and a receptor may be switched.

In certain contexts, an “antibody” refers to a protein-based molecule that is naturally produced by animals in response to the presence of a protein or other molecule or that is not recognized by the animal's immune system to be a “self” molecule, i.e. recognized by the animal to be an antigenic foreign molecule. The immune system of the animal will create an antibody to specifically bind the antigen, and thereby using the antigen for targeted degradation. It is well recognized by skilled artisans that the molecular structure of a natural antibody can be synthesized and altered by laboratory techniques. Recombinant engineering can be used to generate fully synthetic antibodies or fragments thereof providing control over variations of the amino acid sequences of the antibody. Thus, the term “antibody” is intended to include natural antibodies, monoclonal antibody, or non-naturally produced synthetic antibodies. These antibodies may have chemical modifications. The term “monoclonal antibodies” refers to a collection of antibodies encoded by the same nucleic acid molecule that are optionally produced by a single hybridoma (or clone thereof) or other cell line, or by a transgenic mammal such that each monoclonal antibody will typically recognize the same antigen. The term “monoclonal” is not limited to any particular method for making the antibody, nor is the term limited to antibodies produced in a particular species, e.g., mouse, rat, etc.

From a structural standpoint, an antibody is a combination of proteins: two heavy chain proteins and two light chain proteins. The heavy chains are longer than the light chains. The two heavy chains typically have the same amino acid sequence. Similarly, the two light chains typically have the same amino acid sequence. Each of the heavy and light chains contain a variable segment that contains amino acid sequences which participate in binding to the antigen. The variable segments of the heavy chain do not have the same amino acid sequences as the light chains. The variable segments are often referred to as the antigen binding domains. The antigen and the variable regions of the antibody may physically interact with each other at specific smaller segments of an antigen often referred to as the “epitope.” Epitopes usually consist of surface groupings of molecules, for example, amino acids or carbohydrates. The terms “variable region,” “antigen binding domain,” and “antigen binding region” refer to that portion of the antibody molecule which contains the amino acid residues that interact with an antigen and confer on the antibody its specificity and affinity for the antigen. Small binding regions within the antigen-binding domain that typically interact with the epitope are also commonly referred to as the “complementarity-determining regions, or CDRs.”

The term “antibody fragment” refers to a peptide or polypeptide which comprises less than a complete, intact antibody. Complete antibodies comprise two functionally independent parts or fragments: an antigen binding fragment known as “Fab,” and a carboxy terminal crystallizable fragment known as the “Fc” fragment. The Fab fragment includes the first constant domain from both the heavy and light chain (CH1 and CL1) together with the variable regions from both the heavy and light chains that bind the specific antigen. Each of the heavy and light chain variable regions includes three complementarity determining regions (CDRs) and framework amino acid residues which separate the individual CDRs. The Fc region comprises the second and third heavy chain constant regions (CH2 and CH3) and is involved in effector functions such as complement activation and attack by phagocytic cells. In some antibodies, the Fc and Fab regions are separated by an antibody “hinge region,” and depending on how the full-length antibody is proteolytically cleaved, the hinge region may be associated with either the Fab or Fc fragment. For example, cleavage of an antibody with the protease papain results in the hinge region being associated with the resulting Fc fragment, while cleavage with the protease pepsin provides a fragment wherein the hinge is associated with both Fab fragments simultaneously. Because the two Fab fragments are in fact covalently linked following pepsin cleavage, the resulting fragment is termed the F(ab′)2 fragment.

The term “mesenchymal stromal cells” refers to the subpopulation of fibroblast or fibroblast-like nonhematopoietic cells with properties of plastic adherence and capable of in vitro differentiation into cells of mesodermal origin which may be derived from bone marrow, adipose tissue, umbilical cord (Wharton's jelly), umbilical cord perivascular cells, umbilical cord blood, amniotic fluid, placenta, skin, dental pulp, breast milk, and synovial membrane, e.g., fibroblasts or fibroblast-like cells with a clonogenic capacity that can differentiate into several cells of mesodermal origin, such as adipocytes, osteoblasts, chondrocytes, skeletal myocytes, or visceral stromal cells. The term, “mesenchymal stem cells” refers to the cultured (self-renewed) progeny of primary mesenchymal stromal cell populations.

The term “sample” is used in its broadest sense. In one sense it can refer to a plant cell or tissue. In another sense, it is meant to include a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention. The term “sample” is used in its broadest sense. In one sense it can refer to a biopolymeric material. In another sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples.

The term “purified” refers to molecules, either nucleic or amino acid sequences, that are removed from their natural environment, isolated, or separated. An “isolated nucleic acid sequence” is therefore a purified nucleic acid sequence. “Substantially purified” molecules are at least 60% free, preferably at least 75% free, and more preferably at least 90% free from other components with which they are naturally associated by weight.

The term “fusion” when used in reference to a polypeptide refers to a chimeric protein containing a protein of interest joined to an exogenous protein fragment (the fusion partner). The fusion partner may serve various functions, including enhancement of solubility of the polypeptide of interest, as well as providing an “affinity tag” to allow purification of the recombinant fusion polypeptide from a host cell or from a supernatant or from both. If desired, the fusion partner may be removed from the protein of interest after or during purification.

The term “affinity chromatography” refers to a method of separating a biochemical mixture based on specific interaction between binding partners for example, an antigen and antibody, enzyme and substrate, receptor and ligand, lectin and polysaccharide, nucleic acid and complementary base sequence, hormone and receptor, avidin and biotin, glutathione and GST fusion protein. A stationary phase is modified with molecules that specifically bind a target molecule. The target molecules interact with the stationary phase which separates the target molecule from the undesired material which will not interact. The unbound molecules are washed away from the stationary phase. The desired targets are released from the stationary phase in the presence of an eluting solvent. Binding to the solid phase may be achieved by column chromatography whereby the solid medium is packed onto a column. A sample, liquids, and elute are passed through the column. Alternatively, binding may be achieved using a batch treatment, for example, by adding the sample to the solid phase in a vessel, mixing, separating the solid phase, removing the liquid phase, washing, re-centrifuging, adding the elution buffer, re-centrifuging and removing the elute.

The term “nucleic acid” refers to a polymer of nucleotides, or a polynucleotide, as described above. The term is used to designate a single molecule, or a collection of molecules. Nucleic acids may be single stranded or double stranded and may include coding regions and regions of various control elements.

A “heterologous” nucleic acid sequence or peptide sequence refers to a nucleic acid sequence or peptide sequence that do not naturally occur, e.g., because the whole sequences contain a segment from other plants, bacteria, viruses, other organisms, or joinder of two sequences that occur the same organism but are joined together in a manner that does not naturally occur in the same organism or any natural state.

The term “recombinant” when made in reference to a nucleic acid molecule refers to a nucleic acid molecule which is comprised of segments of nucleic acid joined together by means of molecular biological techniques provided that the entire nucleic acid sequence does not occurring in nature, i.e., there is at least one mutation in the overall sequence such that the entire sequence is not naturally occurring even though separately segments may occurring in nature. The segments may be joined in an altered arrangement such that the entire nucleic acid sequence from start to finish does not naturally occur. The term “recombinant” when made in reference to a protein or a polypeptide refers to a protein molecule that is expressed using a recombinant nucleic acid molecule.

The terms “vector” or “expression vector” refer to a recombinant nucleic acid containing a desired coding sequence and appropriate nucleic acid sequences necessary for the expression of the operably linked coding sequence in a particular host organism or expression system, e.g., cellular or cell-free. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

Protein “expression systems” refer to in vivo (e.g. cell) and in vitro (cell free) systems. Systems for recombinant protein expression typically utilize cells transfecting with a DNA expression vector that contains the template. The cells are cultured under conditions such that they translate the desired protein. Expressed proteins are extracted for subsequent purification. In vivo protein expression systems using prokaryotic and eukaryotic cells are well known. Proteins may be recovered using denaturants and protein-refolding procedures. For the purpose of expression system, the term “cell” is not intended to include a pluripotent embryonic stem cell. In vitro (cell-free) protein expression systems typically use translation-compatible extracts of whole cells or compositions that contain components sufficient for transcription, translation and optionally post-translational modifications such as RNA polymerase, regulatory protein factors, transcription factors, ribosomes, tRNA cofactors, amino acids and nucleotides. In the presence of an expression vectors, these extracts and components can synthesize proteins of interest. Cell-free systems typically do not contain proteases and enable labeling of the protein with modified amino acids. Some cell free systems incorporated encoded components for translation into the expression vector. See, e.g., Shimizu et al., Cell-free translation reconstituted with purified components, 2001, Nat. Biotechnol., 19, 751-755 and Asahara & Chong, Nucleic Acids Research, 2010, 38(13): e141, both hereby incorporated by reference in their entirety.

A “selectable marker” is a nucleic acid introduced into a recombinant vector that encodes a polypeptide that confers a trait suitable for artificial selection or identification (report gene), e.g., beta-lactamase confers antibiotic resistance, which allows an organism expressing beta-lactamase to survive in the presence antibiotic in a growth medium. Another example is thymidine kinase, which makes the host sensitive to ganciclovir selection. It may be a screenable marker that allows one to distinguish between wanted and unwanted cells based on the presence or absence of an expected color. For example, the lac-z-gene produces a beta-galactosidase enzyme that confers a blue color in the presence of X-gal (5-bromo-4-chloro-3-indolyl-β-D-galactoside). If recombinant insertion inactivates the lac-z-gene, then the resulting colonies are colorless. There may be one or more selectable markers, e.g., an enzyme that can complement to the inability of an expression organism to synthesize a particular compound required for its growth (auxotrophic) and one able to convert a compound to another that is toxic for growth. URA3, an orotidine-5′ phosphate decarboxylase, is necessary for uracil biosynthesis and can complement ura3 mutants that are auxotrophic for uracil. URA3 also converts 5-fluoroorotic acid into the toxic compound 5-fluorouracil. Additional contemplated selectable markers include any genes that impart antibacterial resistance or express a fluorescent protein. Examples include, but are not limited to, the following genes: amp^{r}, cam^{r}, tet^{r}, blasticidin^{r}, neo^{r}, hyg^{r}, abx^{r}, neomycin phosphotransferase type II gene (nptII), p-glucuronidase (gus), green fluorescent protein (gfp), egfp, yfp, mCherry, p-galactosidase (lacZ), lacZa, lacZAM15, chloramphenicol acetyltransferase (cat), alkaline phosphatase (phoA), bacterial luciferase (luxAB), bialaphos resistance gene (bar), phosphomannose isomerase (pmi), xylose isomerase (xylA), arabitol dehydrogenase (atlD), UDP-glucose:galactose-1-phosphate uridyltransferase (galT), feedback-insensitive α subunit of anthranilate synthase (OASA1D), 2-deoxyglucose (2-DOGR), benzyladenine-N-3-glucuronide, E. coli threonine deaminase, glutamate 1-semialdehyde aminotransferase (GSA-AT), D-amino acidoxidase (DAAO), salt-tolerance gene (rstB), ferredoxin-like protein (pflp), trehalose-6-P synthase gene (AtTPS1), lysine racemase (lyr), dihydrodipicolinate synthase (dapA), tryptophan synthase beta 1 (AtTSB1), dehalogenase (dhlA), mannose-6-phosphate reductase gene (M6PR), hygromycin phosphotransferase (HPT), and D-serine ammonialyase (dsdA).

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “label receptor” refers to incorporation of a heterologous polypeptide in the receptor. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I) fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

In certain embodiments, the disclosure relates to recombinant polypeptides comprising sequences disclosed herein or variants or fusions thereof wherein the amino terminal end or the carbon terminal end of the amino acid sequence are optionally attached to a heterologous amino acid sequence, label, or reporter molecule.

In certain embodiments, the disclosure relates to the recombinant vectors comprising a nucleic acid encoding a polypeptide disclosed herein or chimeric protein thereof.

In certain embodiments, the recombinant vector optionally comprises a mammalian, human, insect, viral, bacterial, bacterial plasmid, yeast associated origin of replication or gene such as a gene or retroviral gene or lentiviral LTR, TAR, RRE, PE, SLIP, CRS, and INS nucleotide segment or gene selected from tat, rev, nef, vif, vpr, vpu, and vpx or structural genes selected from gag, pol, and env.

In certain embodiments, the recombinant vector optionally comprises a gene vector element (nucleic acid) such as a selectable marker region, lac operon, a CMV promoter, a hybrid chicken B-actin/CMV enhancer (CAG) promoter, tac promoter, T7 RNA polymerase promoter, SP6 RNA polymerase promoter, SV40 promoter, internal ribosome entry site (IRES) sequence, cis-acting woodchuck post regulatory element (WPRE), scaffold-attachment region (SAR), inverted terminal repeats (ITR), FLAG tag coding region, c-myc tag coding region, metal affinity tag coding region, streptavidin binding peptide tag coding region, polyHis tag coding region, HA tag coding region, MBP tag coding region, GST tag coding region, polyadenylation coding region, SV40 polyadenylation signal, SV40 origin of replication, Col E1 origin of replication, f1 origin, pBR322 origin, or pUC origin, TEV protease recognition site, loxP site, Cre recombinase coding region, or a multiple cloning site such as having 5, 6, or 7 or more restriction sites within a continuous segment of less than 50 or 60 nucleotides or having 3 or 4 or more restriction sites with a continuous segment of less than 20 or 30 nucleotides.

### Endonuclease V Fusion Peptides and Kits Related Thereto

The term “endonuclease V (EndoV)” refers to a DNA repair enzyme which hydrolyzes the second phosphodiester bond 3′ from a deaminated nucleotide base such as inosine, xanthosine, oxanosine, and uridine. EndoV family proteins exist in eubacteria, archaea, and eukaryotes. Eukaryotic EndoV homologues are typically larger prokaryotic homologues. See Feng et al., Biochemistry, 2005, 44, 11486-11495. The amino acid sequence of Escherichia coli endonuclease V is reported as NCBI Reference Sequence: WP_000362388.1. (SEQ ID NO: 2)

MDLASLRAQQIELASSVIREDRLDKDPPDLIAGADVGFEQGGEVTRAAM
VLLKYPSLELVEYKVARIATTMPYIPGFLSFREYPALLAAWEMLSQKPD
LVFVDGHGISHPRRLGVASHFGLLVDVPTIGVAKKRLCGKFEPLSSEPG
ALAPLMDKGEQLAWVWRSKARCNPLFIATGHRVSVDSALAWVQRCMKGY
RLPEPTRWADAVASERPAFVRYTANQP.

In certain embodiments, this disclosure relates to a fusion peptide comprising endonuclease V sequence or Escherichia coli endonuclease V sequence and a heterologous peptide sequence. In certain embodiments, the heterologous peptide sequence is between 4 and 25 amino acids, or between 7 and 25 amino acids, or between 10 and 25 amino acids, or between 4 and 50 amino acids, or between 7 and 50 amino acids, or between 10 and 50 amino acids, greater than 10, 20, or 30 amino acids. In certain embodiments, this disclosure relates to a nucleic acid or vector encoding a fusion peptide disclosed herein in operable combination with a promoter. In certain embodiments, this disclosure relates to a cell or other expression system comprising a nucleic acid or vector disclosed herein.

In certain embodiments, this disclosure relates to kits comprising a fusion peptide comprising an endonuclease V sequence and a heterologous peptide sequence, a specific binding agent conjugated, wherein the specific binding agent binds to the heterologous peptide sequence, and a container or solution comprising calcium ion in the absence of magnesium ion. In certain embodiments the specific binding agent is conjugated to a solid surface, such as a magnetic bead or chromatography resin.

In certain embodiments, the kit comprises a vessel and/or a liquid transfer device such a syringe, pipette, or capillary tube. In certain embodiments, the endonuclease V sequence is an Escherichia coli endonuclease V sequence. In certain embodiments, the specific binding agent is an antibody that binds the heterologous peptide sequence. In certain embodiments, the solution is a pH buffered solution. In certain embodiments, the kit comprises primers for amplifying a segment of RNA. In certain embodiments, the segment of RNA may be known to or suspected to have a position susceptible to A-to-I editing. In certain embodiments, the kit further comprises amplification reagents.

### Methods of Use

This disclosure relates to improved methods of identifying A-to-I RNA edits in a sample. In certain embodiments, this disclosure relates to methods of purifying RNA containing an inosine base comprising the steps of: exposing an RNA sample to endonuclease V or fusion thereof and calcium ions in the absence of magnesium ions providing an RNA and endonuclease V binding complex. In certain embodiments, the methods further comprise purifying the RNA and endonuclease V binding complex from unbound RNA in the sample; separating the RNA from endonuclease V providing separated RNA; sequencing the separated RNA; and identifying positions in the RNA sequences wherein A-to-I edits occur. In certain embodiments, the RNA is derived from a cell.

In certain embodiments, this disclosure relates to methods of isolating RNA enriched with an inosine base comprising, mixing an endonuclease V, calcium ions in the absence of magnesium ions, and an sample comprising RNA with an inosine base, under conditions such that the endonuclease V binds to the RNA forming an endonuclease V and RNA complex; purifying the endonuclease V and RNA complex; and releasing the RNA from the complex providing isolated RNA enriched with an inosine base. In certain embodiments, the endonuclease V is Escherichia coli endonuclease V. In certain embodiments, said purifying the endonuclease V and RNA complex comprises separating the endonuclease V and RNA complex from RNA that does not substantially contain an inosine base in the sample.

In certain embodiments, said purifying the endonuclease V and RNA complex comprises mixing the endonuclease V and RNA complex with a specific binding agent that binds with a target peptide conjugated to the endonuclease V such that an endonuclease V, RNA, and specific binding agent complex is formed and purifying the endonuclease V, RNA, and specific binding agent complex. In certain embodiments, the specific binding agent is an antibody, and the target peptide comprises an epitope of the antibody.

In certain embodiments, said purifying the endonuclease V and RNA complex comprises mixing the endonuclease V and RNA complex with a specific binding agent that binds with a ligand conjugated to the endonuclease V or binds endonuclease V such that an endonuclease V, RNA, and specific binding agent complex is formed and purifying the endonuclease V, RNA, and specific binding agent complex. In certain embodiments, the specific binding agent is an antibody, and the ligand comprises an epitope of the antibody.

In certain embodiments, the specific binding agent is conjugated to a magnetic bead.

In certain embodiments, said purifying the endonuclease V, RNA, and specific binding agent complex comprises exposing the magnetic bead to a magnetic field such that movement of the bead is held by the magnetic field and moving the magnetic field away from the sample or moving the sample away from the magnetic field.

In certain embodiments, any of the methods disclosed herein further comprise the step of releasing the RNA from the endonuclease V, RNA, and specific binding agent complex providing isolate RNA comprising an inosine base.

In certain embodiments, any of the methods disclosed herein further comprise sequencing the isolated RNA comprising an inosine base.

In certain embodiments, the RNA comprising an inosine base is single stranded or double stranded.

In certain embodiments, any of the methods disclosed herein further comprise the step of mixing the RNA comprising an inosine base with glyoxal.

In certain embodiments, for any of the methods disclosed herein calcium ions are at a concentration of 0.1 to 20 mM, or 0.01 to 20 mM, or 0.1 to 10 mM, 0.01 to 10 mM.

In certain embodiments, for any of the methods disclosed herein the Escherichia coli endonuclease V is a concentration of 0.1 to 5 nM, or 0.01 to 5 nM, or 0.1 to 10 nM, or 0.01 to 10 nM, or 0.1 to 20 nM, or 0.01 to 20 nM.

In certain embodiments, this disclosure relates to methods of purifying and identifying cellular RNA comprising an inosine base comprising, isolating RNA from a cell; breaking the isolated RNA into RNA fragments; mixing the RNA fragments with glyoxal providing a sample of single stranded RNA comprising an inosine base; mixing an endonuclease V, calcium ions in the absence of magnesium ions, and the sample of single stranded RNA comprising an inosine base, under conditions such that the endonuclease V bind to the RNA forming an endonuclease V and RNA complex; purifying the endonuclease V and RNA complex; and releasing the RNA from the endonuclease V, and RNA complex providing isolated cellular RNA comprising an inosine base.

In certain embodiments, said breaking the isolated RNA into RNA fragments results in fragments having an average of less than 500 contiguous nucleotides in length. In certain embodiments, the method further comprises removing glyoxal from the isolated cellular RNA comprising an inosine base. In certain embodiments, the method further comprises sequencing the isolating cellular RNA comprising an inosine base.

In certain embodiments, said purifying the endonuclease V and RNA complex comprises mixing the endonuclease V and RNA complex with a specific binding agent that specifically binds endonuclease V or binds with a ligand conjugated to the endonuclease V such that an endonuclease V, RNA, and specific binding agent complex is formed and purifying the endonuclease V, RNA, and specific binding agent complex.

In certain embodiments, the specific binding agent is an antibody, and the ligand comprises an epitope of the antibody.

In certain embodiments, the specific binding agent is conjugated to a magnetic bead or other solid surface.

In certain embodiments, said purifying the endonuclease V, RNA, and specific binding agent complex comprises exposing the magnetic bead to a magnetic field such that movement of the bead is held by the magnetic field and moving the magnetic field away from the sample or moving the sample away from the magnetic field.

In certain embodiments, purifying is a chromatography method. In certain embodiments, the purifying method comprises securing a specific binding agent to a solid surface, wherein the specific binding agent specifically binds endonuclease V or binds with a ligand conjugated to the endonuclease V, and the endonuclease V and RNA complex are contained in a liquid solution passed over the solid surface whereby the endonuclease V and RNA complex is bound to the specific binding agent on the solid surface, wherein RNA not containing the inosine base flows past the surface providing a purified endonuclease V, RNA, and specific binding agent complex on the surface, and mixing the endonuclease V, RNA, and specific binding agent complex on the surface with releasing agents that separates the RNA from binding to the endonuclease V, thereby providing purified RNA with an inosine base.

In certain embodiments, the cell is a neuron, blood cell, bone marrow cell, brain cell, urine cell, cancer cell, mesenchymal stem cell, or fibroblast.

Selective Enrichment of A-to-I Edited Transcripts from Cellular RNA Using Endonuclease V

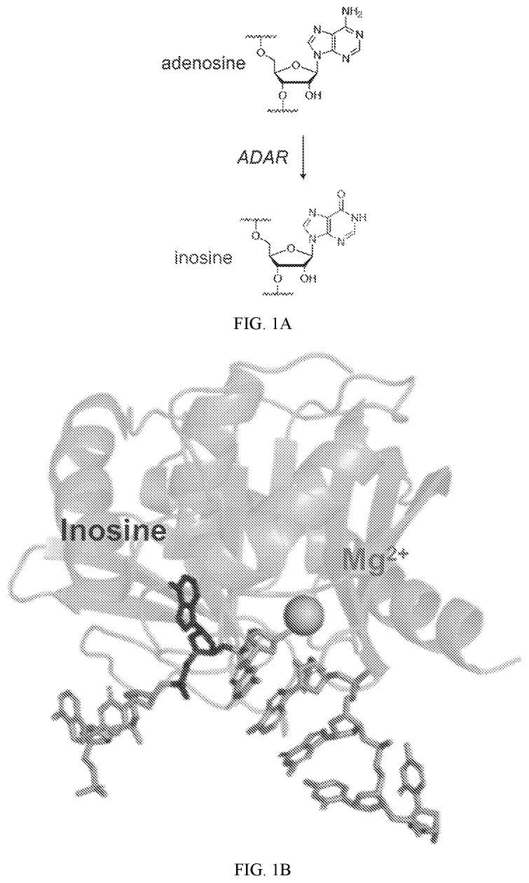

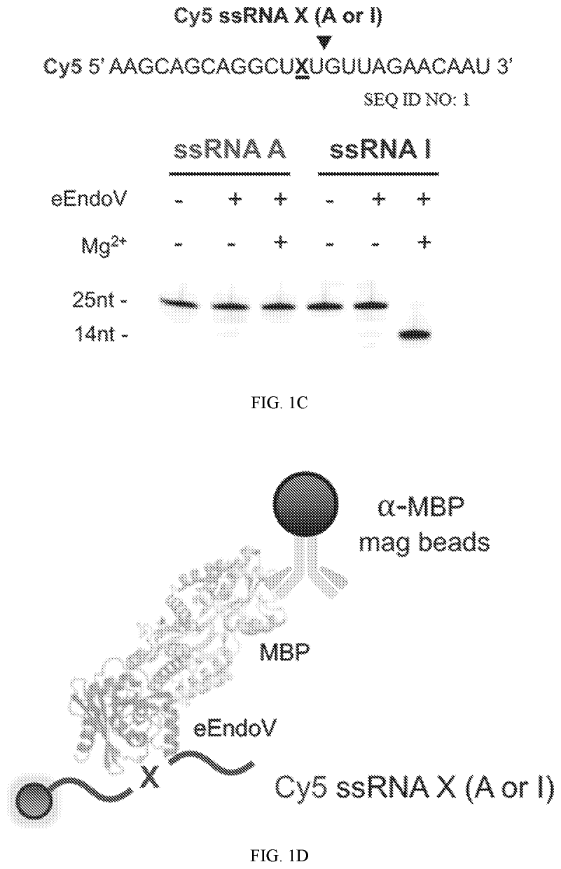

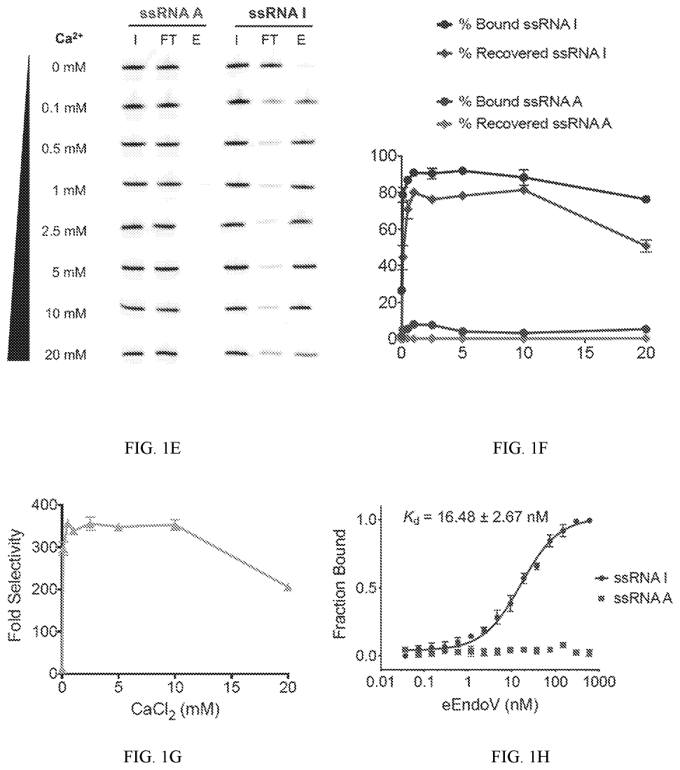

Adenosine-to-inosine (A-to-I) RNA editing is an abundant post-transcriptional modification found in animals. Catalyzed by adenosine deaminases acting on RNAs (ADARs), this reaction alters both the chemical structure and hydrogen bonding patterns of the nucleobase (FIG. 1A). Inosines preferentially base pair with cytidine, effectively recoding these sites as guanosine. A-to-I editing is widespread across the transcriptome and present in most types of RNA. In mRNA, these sites are primarily found in repetitive and untranslated regions, affecting transcript stability, localization, and interactions with cellular pathways. mRNA editing sites can also augment transcript splicing and directly alter amino acid sequences in open reading frames. Additionally, A-to-I editing modulates the target specificities and biogenesis of small-interfering RNAs (siRNAs) and microRNAs (miRNAs), in turn affecting global gene expression patterns and overall cellular behavior. A-to-I editing continues to be implicated in a variety of critical biological processes including embryogenesis, stem cell differentiation, and innate cellular immunity. Dysfunctional A-to-I editing has also been linked with numerous disease processes such as autoimmune disorders and several types of cancer. Recent work has also demonstrated A-to-I editing as a vital driver of human brain development and overall nervous system function, and dysregulated activity has similarly been implicated in a variety of neurological disorders including epilepsy, amyotrophic lateral sclerosis, glioblastoma, schizophrenia, autism, and Alzheimer's disease.

Robust identification and detection of A-to-I sites is vital to understanding these broader biological roles, regulation dynamics, and relationships with disease. Because inosine is decoded as guanosine during reverse transcription, most contemporary methods utilize high-throughput RNA sequencing (RNA-seq) to identify editing sites from A-G transitions. While seemingly simple, the natural complexity of cellular RNA and large dynamic ranges between individual transcripts renders RNA-seq inherently susceptible to random sampling and technical variability, making it challenging to consistently capture and detect RNA editing events, especially in light of the relative scarcity of A-to-I editing sites. Although ˜5 million sites have been identified across the transcriptome, inosine content is low in the context of total cellular RNA, appearing in relatively few actual reads in RNA-seq datasets. This can be attributed to the fact that many key edited transcripts are expressed at low copy number. Moreover, the editing rates at individual sites can be very low or only conditionally active, and can differ significantly across cell and tissue types, individual organisms, developmental stages, and disease states. Because of these technical challenges in RNA-seq, stringent bioinformatic analyses are also important for accurate detection, and extensive computational screening is needed to separate true A-to-I sites from sequencing errors, single-nucleotide polymorphisms (SNPs), somatic mutations, or spurious chemical alterations in RNA.

Enriching A-to-I edited transcripts prior to sequencing addresses challenges by depleting RNAs that otherwise lead to “wasted” sequencing reads while also helping to validate the editing sites that are observed. Effective methods to specifically target and isolate inosine in RNA have not previously been elucidated. Polyclonal antibodies for isolating modified tRNAs were also found to cross-react with several other nucleobases. Inosine chemical labeling strategies were explored using acrylamide and acrylonitrile derivatives. However, these reagents irreversibly modify transcripts with adducts that inhibit reverse transcription, and inherently display off-target reactivity with pseudouridine and uridine, limiting enrichment efficiency.

Endonuclease V (EndoV) was identified as a conserved nucleic acid repair enzyme capable of recognizing and binding to inosine. In prokaryotes, EndoV cleaves downstream of inosine lesions resulting from oxidative damage in DNA to promote base excision repair. In humans and other metazoans, EndoV has now been implicated in the metabolism of A to-I edited RNAs. If cleavage activity could be selectively suppressed without compromising recognition and binding, then EndoV could be leveraged for enriching A-to-I edited RNAs. Escherichia coli EndoV (eEndoV) is both specific and highly active toward inosine in single-stranded RNA (ssRNA) and exhibited minimal sequence bias. E. coli EndoV was explored for the pulldown and enrichment of A-to-I edited transcripts. EndoVIPER-seq (endonuclease V inosine precipitation enrichment sequencing) is an effective approach to bind and isolate inosine-containing transcripts prior to RNA-sequencing, producing significantly improved coverage and detection of A-to-I editing sites in cellular RNA.

Structural analyses have revealed that EndoV requires Mg²⁺ as a cofactor for inosine recognition and strand scission (FIG. 1B). Experiments were performed to determine whether supplementing eEndoV with Ca²⁺ would enable enrichment of inosine containing RNAs from cellular RNA. A pair of Cy5-labeled oligoribonucleotides were synthesized having either A or I in a defined position and evaluated eEndoV activity in the presence of both cations. Specific cleavage activity towards inosine was observed in ssRNA (RNA I) when benchmarked against a non-edited control (ssRNA A) (FIG. 1C). The effect of Ca²⁺ supplementation was evaluated on the ability of eEndoV to bind and isolate inosine-containing ssRNA. The recombinant eEndoV was fused to a maltose-binding protein (MBP) tag, enabling implementation a magnetic workflow using anti-MBP functionalized beads herein after referred to as EndoVIPER (endonuclease V inosine precipitation enrichment, FIG. 1D). This method was used to attempt pulldown both ssRNA A and ssRNA I in the presence of variable amounts of Ca²⁺, while monitoring the initial, unbound (flow-through), and elution fractions after washing (FIG. 1E). Omitting Ca²⁺ produced little binding of either oligonucleotide, supporting the idea that both recognition and cleavage of inosine is mediated through divalent cations. Increasing amounts of Ca²⁺ from 0-10 mM improved binding efficiency substantially, approaching ˜80% recovery with excellent selectivity (˜350-fold over pulldown of RNA A). Additional supplementation beyond 10 mM Ca²⁺ quickly decreased pulldown efficiency and selectivity (FIGS. 1F and 1G). Five (5) mM Ca²⁺ was selected as a suitable concentration for maximizing both recovery and selectivity. These conditions were applied to measure the binding affinity of eEndoV for each RNA substrate using microscale thermophoresis (MST) and observed low nanomolar affinity for ssRNA I and no measurable binding to the ssRNA A control (FIG. 1H).

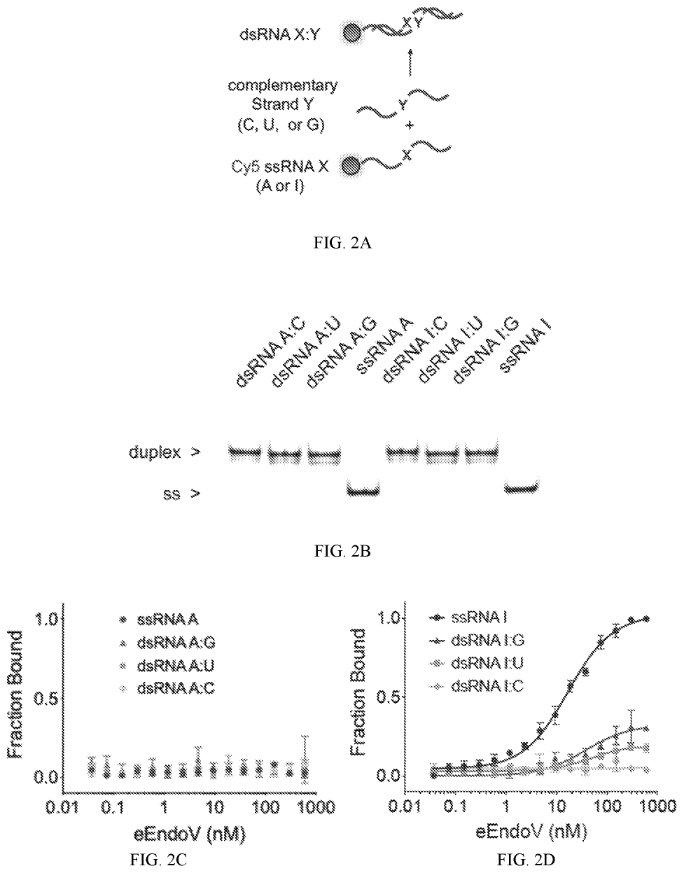

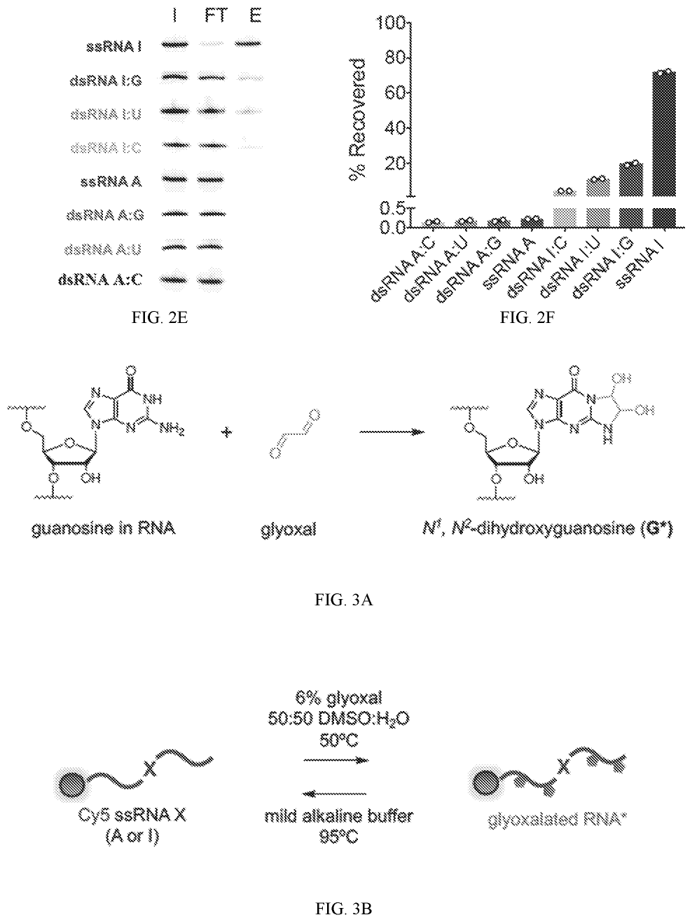

Adenosine deaminases acting on RNAs target structured duplexes. Inosine may reside in the context of dsRNA. EndoV may have difficulty interacting with inosine in these substrates under these binding conditions. Several complementary RNA strands to both ssRNA A and ssRNA I targets were synthesized with differing bases opposite the A/I position. After annealing these strands together (FIGS. 2A and 2B), eEndoV affinity and EndoVIPER performance was assessed with each of the duplex constructs (FIGS. 2C, 2D, and 2F). The enzyme exhibited no detectable binding with any unedited dsRNA A substrates, yet binding affinity towards dsRNA I combinations was highly variable and dependent on the identity of the opposing base in the complementary strand. In particular, a fully complementary duplex (dsRNA I:C) showed virtually no detectable binding by both MST and EndoVIPER (FIGS. 2C, 2D, and 2F), while mismatches ranging from I:U to I:G demonstrated increased binding in both assays. These results are also intriguing in that they are consistent with prior studies of eEndoV on DNA repair together indicating an approximate substrate preference of ssI>>>dsI:G>dsI:U>dsI:C. While interesting, these results posed a challenge to our ultimate goal of designing an unbiased approach to enriching A-to-I edited transcripts from cellular RNA.

The ionic strength of our buffer conditions was reduced, as duplex formation is highly dependent on the presence of cations. Ca²⁺ at 5 mM was choose as the initial concentration for the pulldown step. Experimental results indicate that ˜1-10 mM Ca²⁺ produce similar pulldown efficiencies (FIG. 1G). These tests also employed a standard Tris-buffered saline (19 mM Tris, 137 mM NaCl, 2.7 mM KCl, pH 7.4). It was recognized that lower concentrations of monovalent cations may be tolerated. Conditions assayed having varying concentrations of each cation and found that removing KCl altogether and reducing CaCl2 to 1 mM resulted in highly similar binding affinity and EndoVIPER performance. However, NaCl concentrations below 100 mM resulted in a significant increase in non-specific binding. Despite some promising results, both EndoVIPER and MST analyses indicated that this approach remained insufficient for opening RNA duplexes in our system, and that binding remained highly dependent on structure.

Stronger chemical methods were investigated to fully denature potential dsRNA targets. While several non-covalent denaturants, including formamide and urea, are effective in unfolding stable RNA structures, these also act on proteins. The task is to denature RNA structure while maintaining native eEndoV activity. Covalent methods to reversibly denature RNA prior to EndoVIPER were searched. Such a reagent would ideally provide the following 1) rapidly reacts with RNA under non degrading conditions, 2) stably maintains RNA in a single-stranded state, 3) does not interfere with eEndoV binding, and 4) can be fully removed for downstream sequencing.

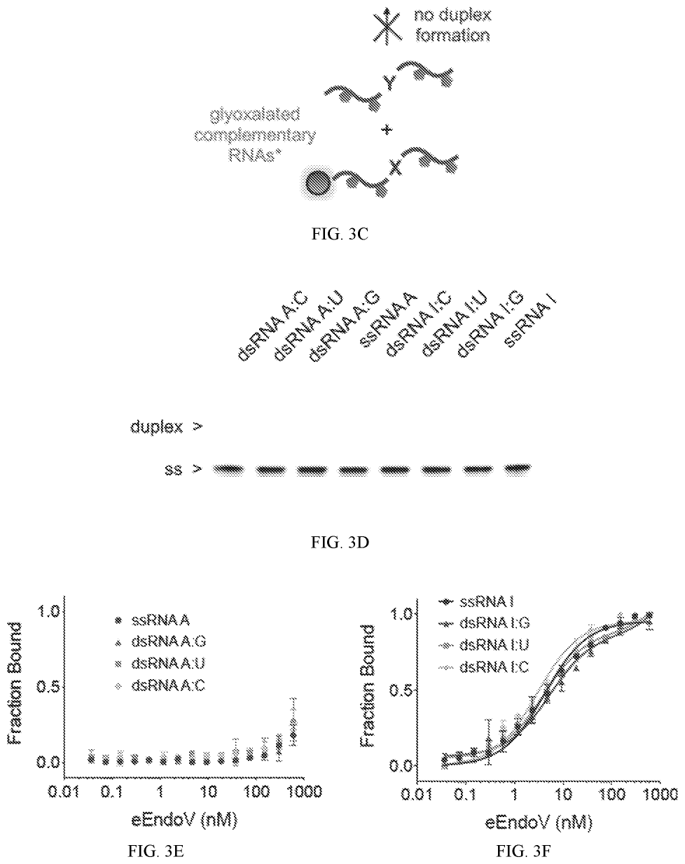

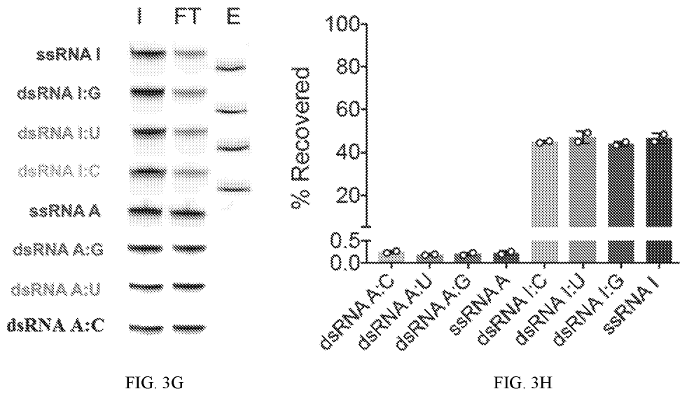

Glyoxal modification of RNA were investigated as this reagent reacts readily with amines on the Watson-Crick-Franklin face to form stable adducts that interfere with base-pairing and RNA secondary structure. While glyoxal can react with A, C, and G, the N¹,N²-dihydroxyguanosine adduct is by far the most stable (FIG. 3A). Importantly, glyoxal does not react with inosine, an observation that has been leveraged to study A-to-I locations. It was uncertain if RNA glyoxalation would be compatible with eEndoV binding. To assess this, ssRNA I and ssRNA A oligoribonucleotides were subjected to glyoxal treatment using. An upward shift in molecular weight was observed when analyzed via 20% PAGE. Binding affinities of eEndoV towards each of the treated RNAs were analyzed. Surprisingly, an improvement in affinity was observed toward glyoxalated ssRNA I, as well as some increased non-specific response towards ssRNA A at higher concentrations of eEndoV. The amount of eEndoV used in the pulldown step was titrated. a clear optimum was observed for both selectivity and efficiency at 100 nM enzyme. Next, the full performance assay was repeated on dsRNA A and I duplex combinations. The target and complementary strands were treated with glyoxal. No duplex formation was observed between glyoxalated RNAs and their complementary strands via 10% native PAGE (FIG. 3D). Binding affinity (FIGS. 3E and 3F) and EndoVIPER efficiency (FIGS. 3G and 3H) were tested on the denatured RNA duplexes. Equivalent performance observed across all RNA I combinations, indicating successful elimination of structural biases in eEndoV binding. While we were encouraged by these results, intermolecular duplexes are relatively easy to disrupt. To ensure that glyoxal treatment prior to EndoVIPER was similarly robust in RNAs having a highly stable internal secondary structure, a hairpin substrate was designed representing a “worst case” RNA target due to its high melting temperature. When this hairpin was chemically denatured with glyoxal, almost identical EndoVIPER performance was observed compared to previous experiments. Together, these data demonstrated that even strong secondary structure could be overcome to enable pulldown with little to no effect on selectivity or enrichment of edited RNAs. However, due to the preferential reaction of glyoxal with guanosine, there was concern about the possibility that G bases adjacent to or near an inosine site could inhibit eEndoV binding. To address this concern, a “G heavy” RNA strand was synthesized as an additional “worst case” test substrate. Nearly identical pulldown and binding affinity was again observed towards this substrate. While there was a slight increase in overall binding affinity when measured by MST, there was no detectable difference in pulldown performance. Together, these experiments demonstrated that the optimized EndoVIPER protocol is robust and displays minimal bias in vitro.

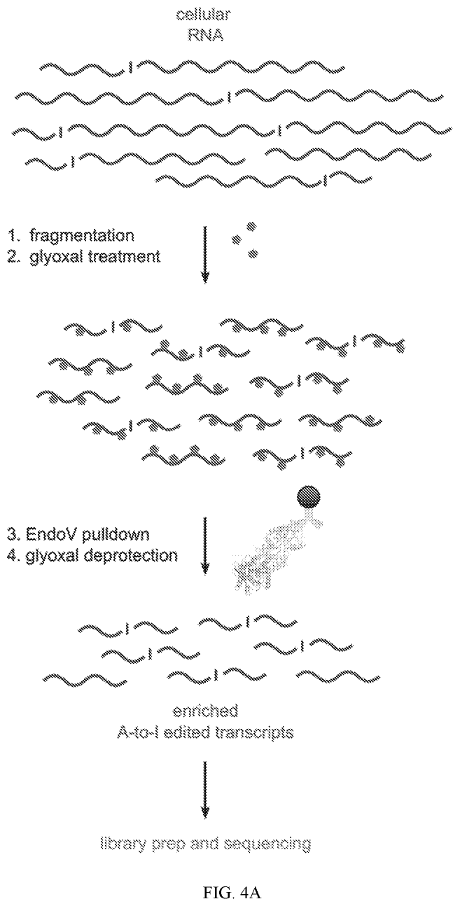

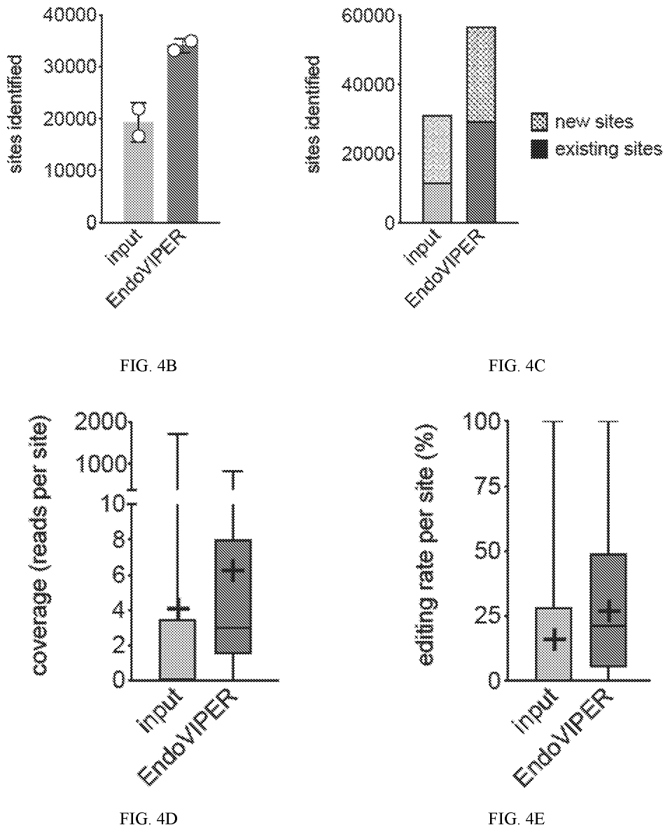

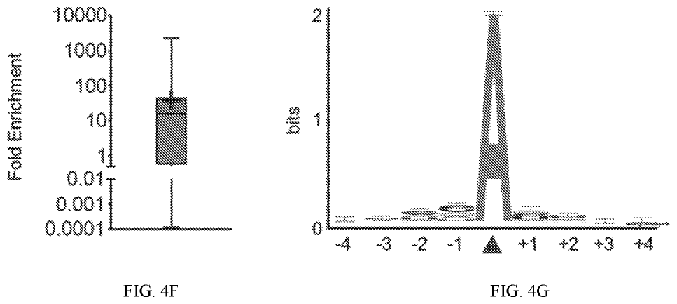

Experiments where performed to test the method in a high-throughput sequencing workflow using cellular RNA. Human brain mRNA was selected to quantify EndoVIPER-seq performance. This tissue is known to have high A-to-I editing activity. Additionally, nervous system tissue is a biologically interesting setting for exploring the enrichment and clinical detection of RNA editing sites crucial for neurological function or indicative of disease. To prepare for high throughput sequencing, RNA material was randomly fragment into smaller strand lengths. fragment sizes of ˜200-500 nt were targeted. It was determined that about a one-minute treatment time with Mg²⁺ at 94° C. was sufficient to yield the desired size distribution. Messenger RNA (mRNA) (2 μg) was fragmented and divided into duplicate “input” and “EndoVIPER” groups (500 ng each). All mRNA samples were then denatured by glyoxal treatment and the EndoVIPER samples subjected to the enrichment workflow (FIG. 4A). After deprotection using heat, all samples were analyzed for size distribution and integrity, confirming that full workflow could be completed without appreciable RNA degradation. Libraries were prepared using about 4 ng of each respective input and EndoVIPER mRNA and proceeded to sequencing. To assess and measure A-to-I editing across samples, a read aligner optimized for RNA editing was employed as well as the specialized REDITools script package and associated filtering steps. From these analyses, it was immediately apparent that the total number of identified sites was significantly higher in EndoVIPER samples (mean 34,084 sites), achieving about 1.8-fold more called A-to-I editing sites compared to input without enrichment (mean 19,308 sites, FIG. 4B). Grouped data was merged and screened against a rigorous annotated database of A-to-I RNA editing (RADAR), REDIPortal, and (DAtabase of RNa EDiting) DARNED databases. An increase in both existing and novel A-to-I locations was observed in EndoVIPER samples (FIG. 4C). The number of newly identified sites was larger than expected in both sample groups (input 19,515 novel positions out of 31,310 total called sites versus EndoVIPER 27,429 novel positions out of 56,744 total called sites). It is worth noting that these databases catalog sites only when detected in several genome-matched donors across many RNA-seq experiments. The experiment utilized commercially available brain mRNA (Takara Bio) isolated and pooled from a small number of donors. Consistent computational assessment was applied between input and EndoVIPER samples. A large increase was reliably observed in the detection of both known and novel editing sites. All inputs were merged and aligned with EndoVIPER datasets (73,578 sites). Both coverage and editing rate were compared at each detected A-to-I location. A significant increase in both metrics across paired sites was observed, indicating that EndoVIPER-seq selectively enriched A-to-I edited RNAs (FIGS. 4D and 4E). On average, 7 to about 38-fold enrichment was observed from read coverage values across all sites, with >75% of these sites displaying equivalent or significantly increased sequencing depth (FIG. 4F). To ensure that eEndoV did not display a sequence context bias, the top 100 most enriched A-to-I sites were compiled, and a sequence motif analysis was performed. No discernable consensus surrounding the editing site was observed in highly enriched transcripts, suggesting minimal EndoVIPER sequence bias (FIG. 4G).

A-to-I editing is critical for normal brain development and function. Editing activity has been identified as a reliable, differential biomarker in a number of neurological disorders. Detection of these pathological editing events is likely to be a component of future RNA-based diagnostic applications, and thus EndoVIPER was employed for monitoring specific editing sites of interest to demonstrate its utility for improving epitranscriptomic characterization. In particular, input and EndoVIPER datasets were applied toward four specific editing site panels, assessing read coverage at 462 editing sites upregulated in postnatal brain development, 403 increased editing events found in autism spectrum disorder, 115 sites with increased editing activity in schizophrenic patients and 31 hyper-edited protein recoding events implicated in glioblastoma carcinogenesis. Read coverage at these sites were directly compared in both input and endoVIPER samples. A consistent overall increase in total read coverage was observed at these positions. These data were also expressed as the number of “edited reads” containing inosine by multiplying coverage with respective calculated editing rate at each site. This trend was similar. Together, these data indicate that EndoVIPER-seq both increased coverage at sites of interest as well as improved specific detection of pathological, edited transcript isoforms, positioning this method as a valuable tool for clinical epitranscriptomics applications.

### EndoVIPER Magnetic IP Assays

For initial binding tests (FIG. 1E), 10 pmol of either RNA I or RNA A was combined with 840 nM eEndoV and variable amounts of CaCl₂) (0, 0.1, 0.5, 1, 2.5, 5, 10 and 20 mM) in a total volume of 50 μL. Final buffer conditions were 19 mM Tris, 137 mM NaCl, 3 mM KCl, 15 μM EDTA, 150 μM DTT, 0.025% Triton X-100, 30 μg/ml BSA, 7% glycerol, pH 7.4. Reactions were incubated at room temperature for 30 min, after which a 3 μL sample (initial, I) was taken and set aside for later analysis. Separately, 70 μL of anti-MBP magnetic bead slurry (New England Biolabs) was washed extensively with a buffer containing 19 mM Tris, 137 mM NaCl, 3 mM KCl, 7% glycerol, and variable amounts of CaCl₂) (0, 0.1, 0.5, 1, 2.5, 5, 10 and 20 mM), pH 7.4. After washing, beads were resuspended in eEndoV-RNA samples and incubated at 25° C. for two hours with end over-end rotation. Magnetic field was applied to the beads and a 3 μL sample (unbound, UB) of the supernatant was saved for later analysis. Beads were washed extensively with respective buffer containing variable amounts of Ca²⁺, and resuspended in 50 μL 19 mM Tris, 137 mM NaCl, 3 mM KCl, 47.5% formamide 0.01% SDS, pH 7.4 and heated to 95° C. for 10 min. Magnetic field was applied and a 3 μL final sample (eluate, E) of the supernatant was taken of each reaction. Collected fractions were analyzed using 10% denaturing PAGE, and gels were imaged using a GE Amersham™ Typhoon™ RGB scanner. Densitometric quantification of bands was performed using ImageJ software. % Bound is expressed as a band intensity ratio of unbound versus initial fractions. % Recovered was defined as the intensity ratio of eluate versus initial fractions. Fold-selectivity was calculated as the ratio of ssRNA I versus ssRNA A recovery percentages. For experiments utilizing RNA duplexes (FIG. 2E), stock constructs were first annealed as described in the later section and 10 pmol of this duplex was used for pulldown using the same protocol as outlined above. For buffer optimization experiments, this pulldown procedure was identical to initial studies above while altering the components of the buffer as outlined in the figure. These optimal formulations are referred to as 1× EndoVIPER (EV) binding buffer (19 mM Tris, 100 mM NaCl, 1 mM CaCl₂), 15 μM EDTA, 150 μM DTT, 0.025% Triton X-100, 30 μg/ml BSA, 7% glycerol, pH 7.4.) and 1× EV wash buffer (19 mM Tris, 100 mM NaCl, 1 mM CaCl₂), 7% glycerol, pH 7.4). To identify optimal eEndoV concentrations, the pulldown procedure was performed by combining 10 pmol of glyoxalated ssRNA I or ssRNA A with 25 nM, 50 nM, 75 nM, 100 nM, 150 nM 200 nM, 400 nM, or 840 nM eEndoV in 1×EV binding buffer and bead-purified with 1×EV wash buffer as described above. Final elution was performed in 50 μL 0.5 M triethylammonium acetate (TEAA) pH 8.6, 47.5% formamide 0.01% SDS (“1×EV elution buffer”) and heated to 95° C. for 10 min, after which samples were analyzed and imaged using 10% denaturing PAGE as described earlier. For pulldown analysis of the hairpin RNA I substrate (hRNA I), 10 pmol of glyoxalated and untreated RNA was incubated with 100 nM eEndoV in 1×EV binding buffer and purified, eluted and analyzed as described earlier using 1×EV wash and EV elution buffers respectively. 10 pmol of “G heavy” RNA strand (G ss RNA I), was tested in an identical manner using 1×EV buffers.

### RNA Duplex Annealing

To assess duplex formation, 100 pmol of each RNA pair (untreated or glyoxalated) were mixed together in 19 mM Tris, 137 mM NaCl, 3 mM KCl, pH 7.4. Mixtures were heated to 95° C. for 5 minutes and slowly cooled to room temperature over the course of approximately 1 hour. Ten pmol of annealed construct was then loaded onto a 10% native non-denaturing polyacrylamide gel and imaged with a GE Amersham™ Typhoon™ RGB scanner.

### Glyoxal Treatment and Deprotection

For initial tests of RNA glyoxalation, 5 ug of ssRNA A or ssRNA I was added to 100 μL of 50% DMSO, 6% glyoxal in nuclease-free water. Samples were reacted for 1 hour at 50° C. and ethanol precipitated. Ten pmol of treated and purified RNA was then analyzed by 10% denaturing PAGE and imaged using a Typhoon™ RGB scanner. To remove glyoxal adducts, 10 pmol of treated and purified RNA was added to 50 μL 0.5 M TEAA pH 8.6, 47.5% formamide, 0.01% SDS and heated to 95° C. for 0, 0.5, 1, 2, 5, 10, 15, and 20 minutes. 5 μL of these reactions were directly analyzed by 20% denaturing page and imaged.

### EndoVIPER-Seq

Two (2) μg human brain mRNA was fragmented for 1 minute at 94° C. using the NEBNext® Magnesium RNA Fragmentation Module (New England Biolabs) and ethanol precipitated. mRNA was then reacted for 1 hour at 50° C. in 100 μL of 50% DMSO, 6% glyoxal in nuclease-free water, followed by ethanol precipitation. Purified pellet was then dissolved in nuclease-free water and quantified using a NanoDrop™ spectrophotometer (Thermo Fisher Scientific). 500 ng of this material was then added to each of two tubes (duplicate “input” samples) containing 30 μL nuclease free water and frozen at −80° C. for later use. For EndoVIPER samples, 500 ng of fragmented, glyoxalated mRNA was added to each of two tubes containing a 250 μL solution of 100 nM eEndoV and 120 units RNasin™ Plus inhibitor (Promega) in 1×EV binding buffer and was incubated at room temperature for 30 minutes. Separately, 300 μL anti-MBP magnetic bead slurry (New England Biolabs) was added to a new microfuge tube and washed extensively with 1×EV wash buffer. After washing, beads were resuspended in the eEndoV-mRNA samples and incubated at room temperature for two hours with end-over-end rotation. A Magnetic field was applied, and the supernatant was discarded. Beads were then washed three times with 500 μL 1×EV wash buffer and then resuspended in 200 μL of 1×EV elution buffer. Bound mRNA was then eluted by heating to 95° C. for 10 min. Residual magnetic beads were removed from the collected supernatant using 0.22 μM microfuge spin filters (Corning™ Costar™), and RNA was purified further with the Monarch™ RNA Cleanup Kit and eluted in nuclease-free water. To ensure full removal of glyoxal adducts, RNA was incubated at 65° C. for 2 hours in 100 μL 50% DMSO in 137 mM NaCl, 2.7 mM KCl, 8 mM Na₂HPO₄, and 2 mM KH₂PO₄, pH 7.4 followed by ethanol precipitation and resuspension in nuclease-free water. Starting mRNA material, fragmented input, and enriched EndoVIPER mRNA were quantified and assessed for size distribution using an Agilent 2100 Bioanalyzer instrument and the Agilent 6000 RNA Pico kit. 8 ng of each input and EndoVIPER RNA replicate was then used to prepare sequencing libraries with the SMARTer® Stranded Total RNA-Seq Kit v2—Pico Input kit (Takara Bio), standard 8-bp i5 and i7 Illumina index barcodes and adapters were added to each library. Libraries were then sequenced using a NextSeq 550 (Illumina) to produce paired end 150-bp reads.

What is claimed is:

1. A method of isolating RNA enriched with an inosine base comprising,
mixing an endonuclease V, calcium ions in the absence of magnesium ions, and a sample comprising RNA comprising an inosine base, under conditions such that the endonuclease V binds to the RNA forming an endonuclease V and RNA complex;
purifying the endonuclease V and RNA complex; and
releasing the RNA from the complex providing isolated RNA enriched with an inosine base.

2. The method of claim 1, wherein said purifying the endonuclease V and RNA complex comprises separating the endonuclease V and RNA complex from RNA that does not contain an inosine base in the sample.

3. The method of claim 1, wherein said purifying the endonuclease V and RNA complex comprises
mixing the endonuclease V and RNA complex with a specific binding agent that binds with a ligand conjugated to the endonuclease V or binds endonuclease V such that an endonuclease V, RNA, and specific binding agent complex is formed and
purifying the endonuclease V, RNA, and specific binding agent complex.

4. The method of claim 3, wherein the specific binding agent is an antibody and the ligand comprises an epitope of the antibody.

5. The method of claim 4, wherein the specific binding agent is conjugated to a magnetic bead.

6. The method of claim 5, wherein said purifying the endonuclease V, RNA, and specific binding agent complex comprises exposing the magnetic bead to a magnetic field such that movement of the bead is held by the magnetic field and moving the magnetic field away from the sample or moving the sample away from the magnetic field.

7. The method of claim 6 further comprising the step of releasing the RNA from the endonuclease V, RNA, and specific binding agent complex providing isolated RNA comprising an inosine base.

8. The method of claim 7 further comprising sequencing the isolate RNA comprising an inosine base.

9. The method of claim 1, wherein the endonuclease V is Escherichia coli endonuclease V.

10. A method of isolating cellular RNA comprising an inosine base comprising,
isolating RNA from a cell;
breaking the isolated RNA into RNA fragments;
mixing the RNA fragments with glyoxal providing a sample of single stranded RNA comprising an inosine base;
mixing an endonuclease V, calcium ions in the absence of magnesium ions, and the sample of single stranded RNA comprising an inosine base, under conditions such that the endonuclease V bind to the RNA forming an endonuclease V and RNA complex;
purifying the endonuclease V and RNA complex; and
releasing the RNA from the endonuclease V, and RNA complex providing isolated cellular RNA comprising an inosine base.

11. The method of claim 10 further comprising removing glyoxal from the isolated cellular RNA comprising an inosine base.

12. The method of claim 11 further comprising sequencing the isolating cellular RNA comprising an inosine base.

13. The method of claim 10, wherein said purifying the endonuclease V and RNA complex comprises
mixing the endonuclease V and RNA complex with a specific binding agent that specifically binds endonuclease V or binds with a ligand conjugated to the endonuclease V such that an endonuclease V, RNA, and specific binding agent complex is formed and
purifying the endonuclease V, RNA, and specific binding agent complex.

14. The method of claim 13, wherein the specific binding agent is an antibody, and the ligand comprises an epitope of the antibody.

15. The method of claim 13, wherein the specific binding agent is conjugated to a magnetic bead.

16. The method of claim 15, wherein said purifying the endonuclease V, RNA, and specific binding agent complex comprises exposing the magnetic bead to a magnetic field such that movement of the bead is held by the magnetic field and moving the magnetic field away from the sample or moving the sample away from the magnetic field.

17. The method of claim 10, wherein the cell is a neuron, blood cell, bone marrow cell, brain cell, urine cell, cancer cell, mesenchymal stem cell, or fibroblast.

18. The method of claim 10, wherein the endonuclease V is Escherichia coli endonuclease V.

19. A fusion peptide comprising Escherichia coli endonuclease V sequence and a heterologous peptide sequence of greater than 10 amino acids.

20-27. (canceled)


SEQUENCE LISTING (2 sequences)

SEQ ID NO: 1 - RNA, 25 nt, Artificial
  Synthetic construct
  n (1)..(25): wherein x is inosine
  misc_feature (13)..(13): n is a, c, g, or u
aagcagcaggcunuguuagaacaau

SEQ ID NO: 2 - protein, 223 aa, Artificial
  Synthetic construct
MDLASLRAQQIELASSVIREDRLDKDPPDLIAGADVGFEQGGEVTRAAMVLLKYPSLELVEYKVARIATTMPYIPGFLSFREYPALLAAWEMLSQKPDLVFVDGHGISHPRRLGVASHFGLLVDVPTIGVAKKRLCGKFEPLSSEPGALAPLMDKGEQLAWVWRSKARCNPLFIATGHRVSVDSALAWVQRCMKGYRLPEPTRWADAVASERPAFVRYTANQP